(19) United States
(12) Patent Application Publication
(10) Pub. No.: US 2018/0177989 A1
(43) Pub. Date: Jun. 28, 2018

(54) VAGINAL REJUVENATION METHODS AND DEVICES

(71) Applicant: Regen Medical Inc., Ft. Lauderdale, FL (US)

(72) Inventor: Mary Ellen Molly PRENTICE, Hobe Sound, FL (US)

(21) Appl. No.: 15/852,365

(22) Filed: Dec. 22, 2017

Related U.S. Application Data
(60) Provisional application No. 62/439,727, filed on Dec. 28, 2016.

(51) Int. Cl. A61M 31/00; A61B 1/303; A61B 1/32

(52) U.S. Cl. CPC A61M 31/002; A61B 2018/00559; A61B 1/32; A61B 1/303

(57) ABSTRACT

Methods and apparatuses for vaginal rejuvenation are provided. A method of vaginal rejuvenation includes providing an injectable solution of amniotic membrane allograft. The solution is injected into a human female in a plurality of locations in a vaginal canal and introitus area to a depth immediately below a vaginal epithelium. A speculum includes at least one longitudinal member extending from an opening along a longitudinal axis and having a wall that curves about the axis having an inner surface and an outer surface, wherein the longitudinal member comprises multiple spaced portals extending through the wall. A kit includes the speculum and the injectable solution of amniotic membrane allograft.

### CLAIM OF PRIORITY UNDER 35 U.S.C. § 119

This application claims priority to U.S. Provisional Application No. 62/439,727 titled “VAGINAL REJUVENATION METHODS AND DEVICES,” filed Dec. 28, 2016, which is assigned to the assignee hereof, and incorporated herein by reference in its entirety.

### INTRODUCTION

The present disclosure generally relates to techniques for vaginal rejuvenation.

### BACKGROUND

Vaginal relaxation is a condition that affects millions of women. Vaginal relaxation is the loss of the optimum structural architecture of the vagina. The vaginal muscles become relaxed and this results in loss of vaginal tone, strength, and tissue integrity. The internal and external diameters of the vaginal canal can increase and the muscles of the perineum become weak and poorly supported. During these circumstances, the vagina is not physiologically optimal, and sexual comfort and gratification can be diminished. Vaginal relaxation can have multiple causes which include but are not limited to childbirth, aging, obesity, injury, previous pelvic surgery, and chronic straining. Less common contributing factors include but are not limited to congenital malformations, sacral nerve disorders, and connective tissue disorders.

Childbirth, and the stretching of the vaginal canal and surrounding tissues during the birthing process is a major cause of vaginal relaxation. Multiple pregnancies further increase the alteration of the vaginal canal and surrounding tissues. The internal and external diameters of the vaginal canal can greatly increase with a significant stretching of the vaginal walls. As well, trauma in the form of tearing in the surrounding muscles and ligaments can occur.

Vaginal relaxation can also take place during the normal aging process. Under these circumstances the vagina is no longer at its physiologically optimum sexual functioning state. The vaginal epithelium becomes very thin, there is a decrease in elasticity and micro tears can occur in the fascia, muscles and ligaments that surround and support the vaginal canal. As stated above, other common causes of vaginal relaxation are obesity, injury and previous pelvic surgery.

Common conditions resulting from vaginal relaxation include but are not limited to: internal vaginal enlargement, enlargement of the vaginal introitus, inadequate pelvic muscle function, incompetence of pelvic fundus, lax vaginal introitus, ligamentous laxity of pelvic region, perineal descent, relaxation of pelvic floor, second degree uterine prolapse, and weakening of the pelvic fundus.

Similar to vaginal relaxation, vulvovaginal atrophy is a common condition that can occur anytime in a woman's lifetime but is most common in the premenopausal and postmenopausal stages. Numerous retrospective studies have evaluated the prevalence of symptoms of vulvovaginal atrophy. Although these studies differ in type of symptoms elicited, study design, and study population, they provide a range of estimates of vulvovaginal atrophy prevalence. In general, the prevalence ranged from about 25% to 45% in the late postmenopausal group. The symptoms can include vaginal dryness, vaginal laxity, painful intercourse, decreased sensation during intercourse, soreness, irritation, and urinary incontinence. Other terms used to describe this condition are atrophic vaginitis and urogenital atrophy.

Healthy vaginal tissues are made of cells embedded or attached to extracellular matrices. The key elements comprising extracellular matrices include collagens, elastins, laminin, fibronectin and proteoglycans. Collectively, all of these elements are important in the overall health, robustness, and resilience of the vaginal epithelium and the surrounding connective tissues. A noticeable decrease in these key elements is a normal part of the aging process and can play a role in the onset of both vaginal relaxation and vulvovaginal atrophy. During menopause, collagen's structure and elasticity are subject to changes at all levels of organization. Changes occur between individual collagen fibers, between collagen and muscle, and between collagen and other matrix elements. Losses in amounts of collagen in the vaginal mucosa may cause the epithelial surface of the vulva and vaginal tissue to thin as well as a degradation of the surrounding connective tissue. This can lead to many of the symptoms experienced in both vaginal relaxation and vulvovaginal atrophy.

### SUMMARY

The following presents a simplified summary of one or more aspects of the invention in order to provide a basic understanding of such aspects. This summary is not an extensive overview of all contemplated aspects, and is intended to neither identify key or critical elements of all aspects nor delineate the scope of any or all aspects. Its purpose is to present some concepts of one or more aspects in a simplified form as a prelude to the more detailed description that is presented later.

The disclosure provides a method of performing a surgical or office based procedure that relates to methods of vaginal rejuvenation including but not limited to introducing amniotic tissue derived collagen, introducing amniotic derived growth factors, cytokines and tissue matrix, and introducing amniotic derived stem cell recruitment potential, with or without the combination of hyaluronic acid, via injection into the vaginal canal and surrounding area which can lead to tightening of the vaginal canal, decreasing the laxity of the vaginal introitus, decreasing the laxity of the vaginal tissue, thickening of the vaginal epithelium, and overall increased strength and integrity of the vaginal canal.

In one aspect, the disclosure provides a method of vaginal rejuvenation. The method may include providing an injectable solution comprising amniotic membrane allograft. The method may include injecting the solution into a human female in a plurality of locations in a vaginal canal and introitus area to a depth immediately below a vaginal epithelium.

The injectable solution may also include hyaluronic acid. The injectable solution may also include saline.

The method may also include inserting a speculum into the vaginal canal. The speculum may include multiple evenly spaced openings extending through at least one blade of the speculum. The injecting may include inserting a needle through a respective one of the evenly spaced openings into each of the plurality of locations. Inserting the needle may include inserting the needle through the respective one of the evenly spaced openings until a stop on the needle abuts a surface of the speculum. The needle may extend to the depth immediately below the vaginal epithelium when the stop abuts the surface of the speculum.

In another aspect, the disclosure provides a speculum. The speculum may include at least one longitudinal member extending from an opening along a longitudinal axis and having a wall that curves about the axis having an inner surface and an outer surface. The longitudinal member may include multiple evenly spaced portals extending through the wall.

In another aspect, the disclosure provides a kit for performing a vaginal rejuvenation procedure. The kit may include a speculum having at least one longitudinal member extending from an opening along a longitudinal axis and having a wall that curves about the axis having an inner surface and an outer surface. The longitudinal member may include multiple evenly spaced portals extending through the wall. The kit may also include an injectable solution comprising amniotic membrane allograft. The kit may also include a syringe and a lubricated vaginal packing.

These and other aspects of the invention will become more fully understood upon a review of the detailed description, which follows.

### BRIEF DESCRIPTION OF THE DRAWINGS

FIG. 1A is an anatomical diagram illustrating first example external locations for injections, according to an aspect of the disclosure.

FIG. 1B is an anatomical diagram illustrating second example external locations for injections, according to an aspect of the disclosure.

FIG. 1C is an anatomical diagram illustrating third example external locations for injections, according to an aspect of the disclosure.

FIG. 1D is an anatomical diagram illustrating fourth example external locations for injections, according to an aspect of the disclosure.

FIG. 2A is an anatomical diagram illustrating first example internal locations for injections, according to an aspect of the disclosure.

FIG. 2B is an anatomical diagram illustrating second example internal locations for injections, according to an aspect of the disclosure.

FIG. 2C is an anatomical diagram illustrating third example internal locations for injections, according to an aspect of the disclosure.

FIG. 2D is an anatomical diagram illustrating fourth example internal locations for injections, according to an aspect of the disclosure.

FIG. 3 is a diagram illustrating an example of a speculum, according to an aspect of the disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known components are shown in block diagram form in order to avoid obscuring such concepts.

Generally described, the disclosure provides for a method of vaginal rejuvenation and an apparatus therefor. The method may include injecting a solution including amniotic membrane allograft into a human female in a plurality of locations in a vaginal canal and introitus area to a depth immediately below a vaginal epithelium. The apparatus may be a speculum that includes multiple portals through the walls of the speculum to position a syringe for injection of the solution in the correct locations to the correct depth. Injection of the solution stimulates repair of tissue while providing nutrients for repairing the tissue.

Amniotic tissue includes multiple extracellular matrix proteins, structural collagen, growth factors, cytokines and other regulatory proteins. Amniotic tissue allografts may contain up to five (5) types of collagen as well as fibronectin, elastin fibers, laminin, and proteoglycans. Aside from being collagen rich, these types of allografts have been shown to promote fibroblast and microvascular endothelial cell proliferation. In addition, amniotic tissue is shown to recruit migration of adult stem cells, including mesenchymal stem cells, adipose derived stem cells, and hematopoietic stem cells. This migration of stem cells has shown modulation of the chronic inflammatory response, the possibility of tissue remodeling, and promotion of physiological and biomechanically compliant neotissue. As well, the cellular signals contained in the amniotic tissue allograft upregulate the production of angiogenic factors. All of these factors combined can lead to increased tone, strength, tissue integrity and overall health of the vaginal canal and surrounding tissues when introduced in an effective application.

An example application method is now described. In an aspect, the method may include preparing the vaginal canal with a cleansing scrub indicated for use in the vaginal canal.

In an aspect, the method may include inserting a speculum into the vaginal canal. The speculum, as described in further detail below, may include multiple portals extending through the walls of the speculum. For example, the portals may be aligned along a line in one blade of the speculum. The speculum may also include portals in a handle portion.

The method may include obtaining an injectable solution including amniotic membrane allograft. The injectable solution may additionally include hyaluronic acid and/or sterile saline. In an aspect, the injectable solution may be packaged with the speculum as a kit. The injectable solution may be drawn into a syringe, as known in the art.

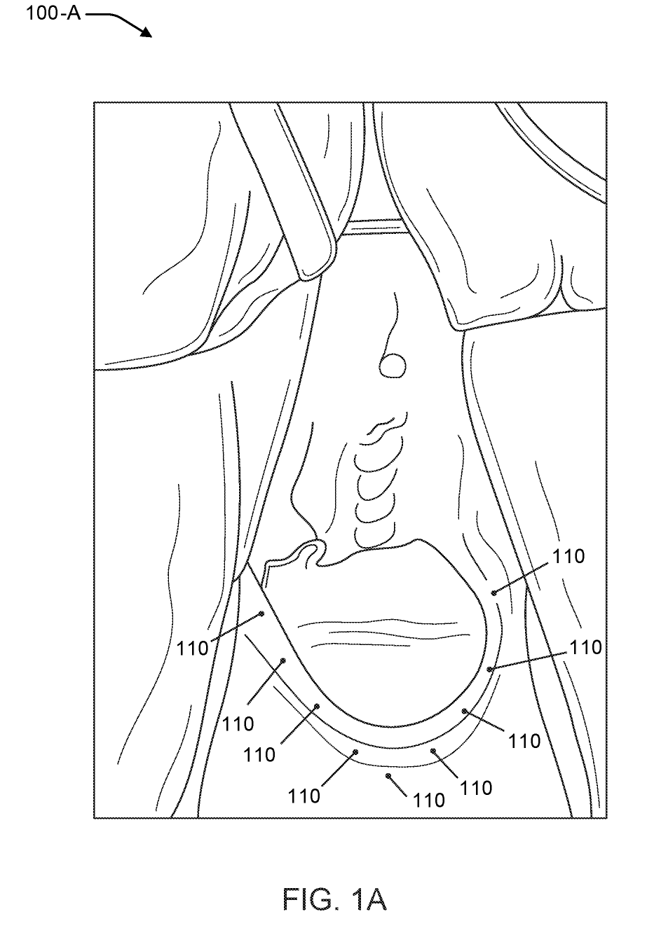

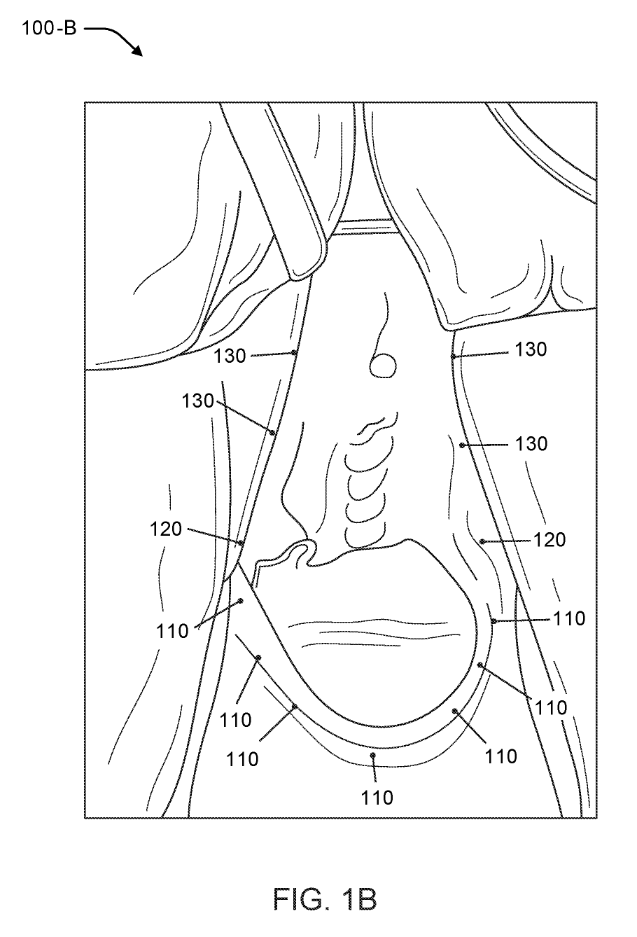

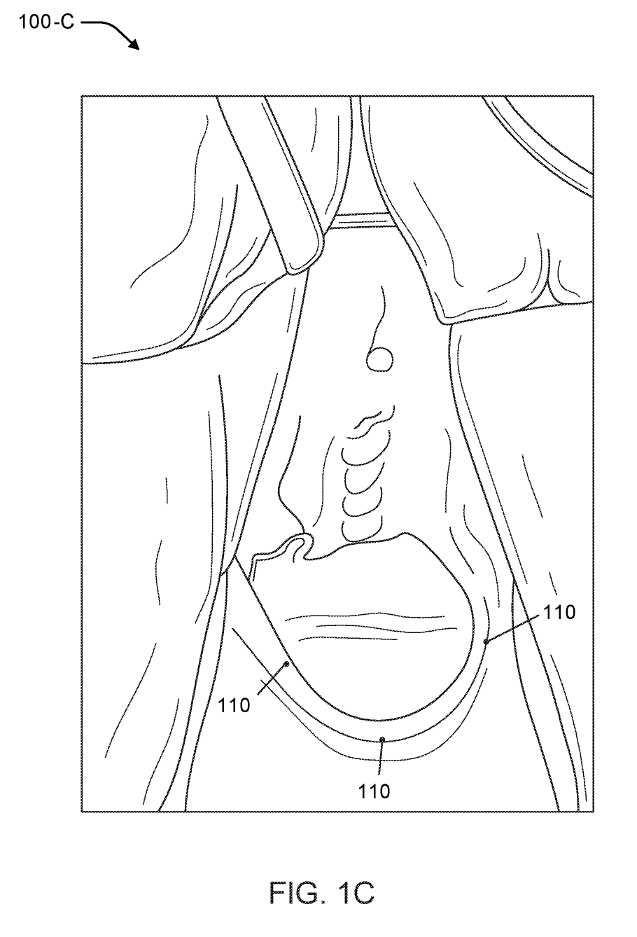

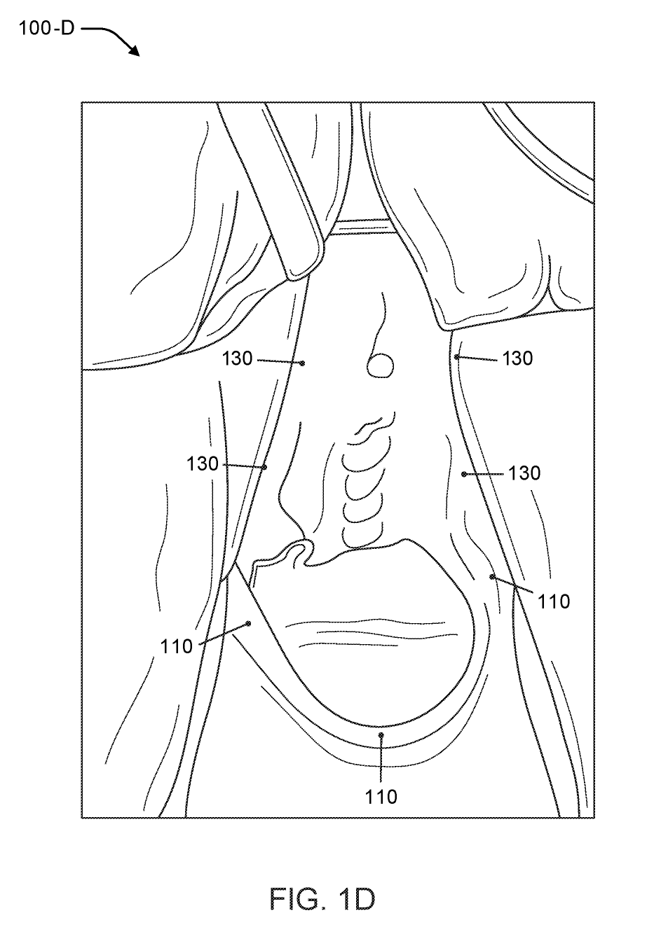

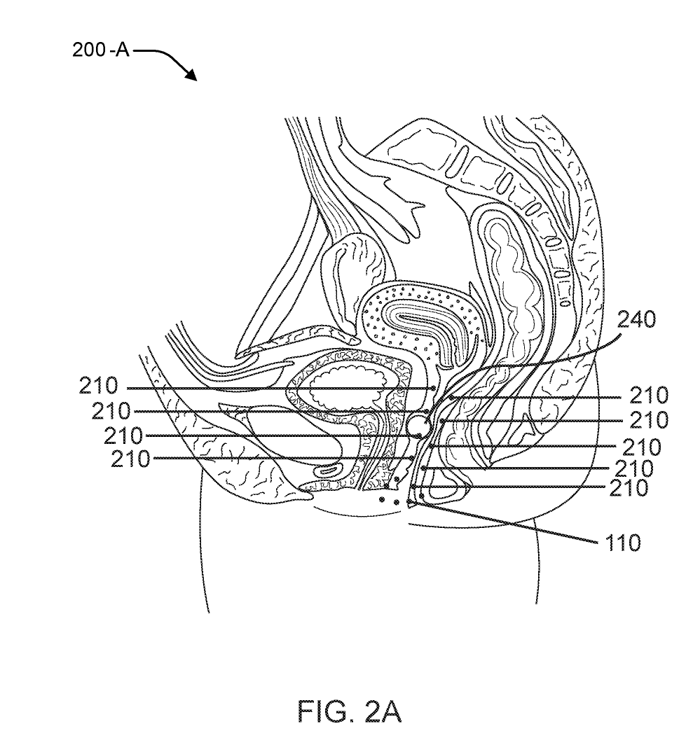

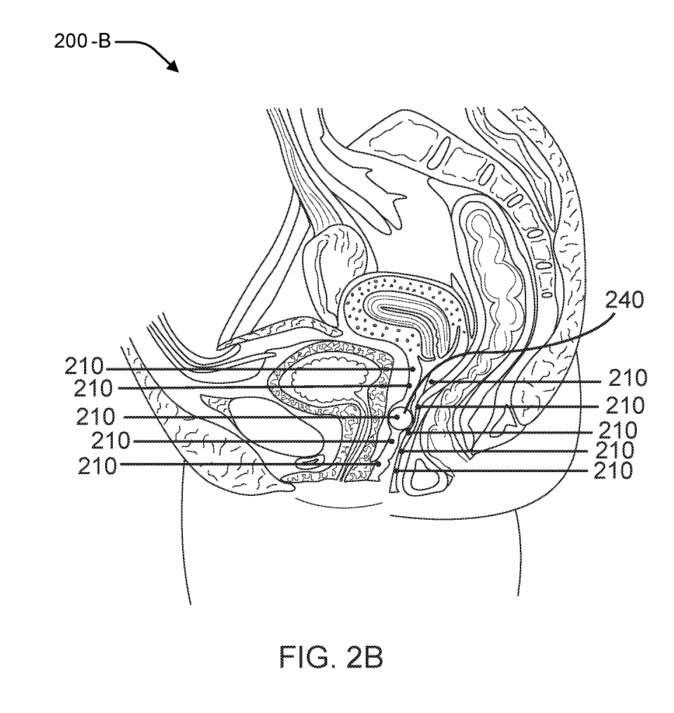

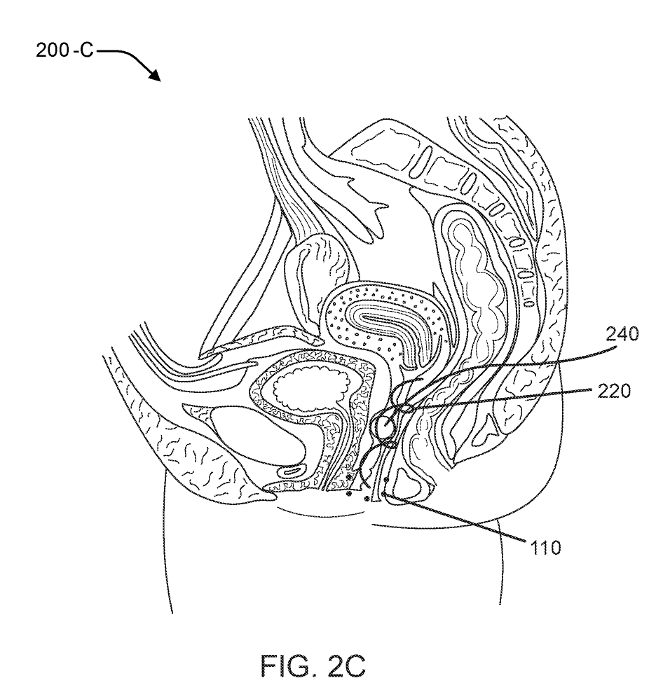

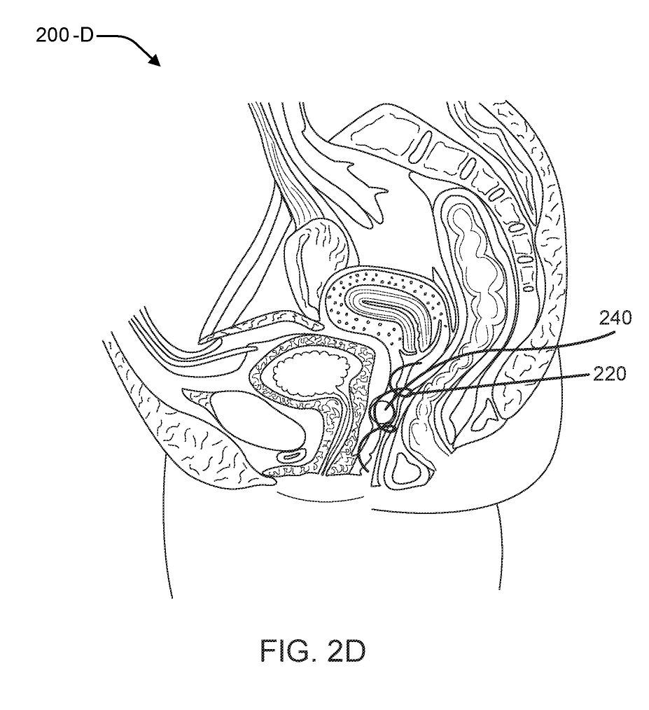

The method may include injecting the solution into a human female in a plurality of locations in a vaginal canal and introitus area to a depth immediately below a vaginal epithelium. The injectable solution may be introduced via a syringe and needle (e.g., 22-25 gauge) just below the vaginal epithelium through multiple injection sites throughout the vaginal canal and introitus area. The vaginal canal including the anterior wall, posterior wall, and lateral sidewalls may be injected with the prepped amniotic membrane solution along the pathways described in further detail below with respect to FIGS. 1A-1D and FIGS. 2A-2D. The needle may be inserted through the vaginal mucosa to a depth just below the epithelial layer into the muscularis. In an aspect, the desired depth may be approximately 1 mm to 10 mm, preferably 1 mm to 8 mm, depending on the anatomy of the patient. For example, in a majority of patients exhibiting vaginal relaxation, the desired depth may be 1 mm to 3 mm. The desired depth may be greater, for example 4 mm to 6 mm, in younger patients. A depth less than 10 mm may avoid puncturing other organs. Small pea sized amounts, (e.g., approximately 0.1 cc) of amniotic solution with or without hyaluronic acid may be injected repeatedly in the patterns and locations illustrated in the figures. The distance between each injection point may be approximately 1 cm-1.5 cm. Although various example injection patterns are illustrated, additional or fewer injections may be adequate based on patient anatomy, degree of symptoms, and amount of blood loss from initial injections.

In an aspect, injecting the solution may be performed with or without use of the speculum. When using the speculum, the needle may be inserted through the portals of the speculum. The portals may be used to determine the locations for the injections. The portals may also serve as a depth guide for the injections. The needle may be inserted until a stop on the needle contacts an inner surface of the speculum. When the speculum is not used, a medical practitioner may select the locations for the injections and determine the depth of the needle. For example, a stop or markings on the needle may be used to gauge the depth.

In an aspect, the method may include injecting an additional 0.2 cc-0.5 cc of solution specifically into the area known as the Gräfenberg Spot or “G-Spot” 240. An injection at the G-Spot 240 may optionally be included with any of the injection patterns described in detail below.

In an aspect, at the conclusion of the injections, the vaginal canal may be packed with a lubricated vaginal packing. The lubricated vaginal packing may be left in place for 15-20 minutes to minimize bleeding.

The above described procedure may be repeated every 45-60 days as needed until the desired result is achieved. This procedure can also be utilized in conjunction with other vaginal rejuvenation procedures such as surgical and nonsurgical laser vaginal rejuvenation, fractional carbon dioxide vaginal rejuvenation, and radio frequency vaginal rejuvenation.

Turning now to the Figures, FIG. 1A is an anatomical diagram 100-A illustrating first example external locations 110 for injections. The external locations 110 are located in the lower external labia and the lower external portions of the vaginal canal. For example, in an aspect, approximately nine (9) injections may be spaced evenly around the entrance to the vaginal canal. Each injection may be approximately 0.1 cc. The distance between each injection point may be approximately 1 cm -1.5 cm.

FIG. 1B is an anatomical diagram 100-B illustrating second example external locations 120 for injections, according to an aspect of the disclosure. In addition to the locations 110, injections may be made at the locations 120 in the upper external portion of the vaginal canal and/or at the locations 130 in the upper external labia. Each injection may be approximately 0.1 cc. The distance between each injection point may be approximately 1 cm-1.5 cm.

FIG. 1C is an anatomical diagram 100-C illustrating third example external locations 110 according to another example. In this example, fewer external injections in comparison to FIG. 1A may be utilized. For example, three (3) external injections may be made in the lower external portions of the vaginal canal and the lower external labia. In an aspect, a larger amount of solution may be used per injection location. For example, approximately 0.2 cc to 0.4 cc may be used. Additionally, the injection locations may be spaced farther apart in comparison to FIG. 1A, for example, approximately 2.0 cm to 4.0 cm apart.

FIG. 1-D is an anatomical diagram 100-D illustrating example external locations for injections, according to an aspect of the disclosure. In this example, fewer external injections in comparison to FIG. 1B may be utilized. For example, three (3) external injections may be made in the locations 110 in the lower external portions of the vaginal canal and the lower external labia and four (4) external injections may be made in the locations 130 in the upper external labia. In an aspect, a larger amount of solution may be used per injection location. For example, approximately 0.2 cc to 0.4 cc may be used. Additionally, the injection locations may be spaced farther apart in comparison to FIG. 1B, for example, approximately 2.0 cm to 4.0 cm apart.

FIG. 2A is an anatomical diagram 200-A illustrating a set of first example internal locations 210 for injections, according to an aspect of the disclosure. The internal locations 210 may be arranged along a substantially straight line on each of the anterior, posterior and lateral sidewalls of the vaginal canal. Additionally, numerous small pea sized amounts, (e.g., approximately 0.1 cc) of amniotic solution with or without hyaluronic acid may injected at external locations 110 in the lower external labia and lower external portion of the vaginal canal.

FIG. 2B is an anatomical diagram 200-B illustrating another set of second example internal locations 210 for injections. In this example, the injections are limited to the vaginal canal only and not the external labia. The distance between each injection point may be approximately 1 cm-1.5 cm.

FIG. 2C is an anatomical diagram 200-C illustrating example internal locations in a spiral pattern 220 for injections, according to an aspect of the disclosure. Numerous small pea sized amounts, (approximately 0.1 cc) of amniotic solution with or without hyaluronic acid may be injected in a spiral pattern 220 starting in the proximal portion of the vaginal canal and progressing distally along the anterior, posterior and lateral sidewalls of the vaginal canal. The distance between each injection point may be approximately 1 cm -1.5 cm.

If using a bivalve speculum, the injections for the spiral pattern 220 may use every fourth portal in the speculum starting with a first portal. The speculum may be turned to access an adjacent wall of the vaginal canal and injections may use every fourth portal in the speculum starting with a second portal that is different than the first portal. The speculum may be rotated again and the injections started at a third portal for the third wall of the vaginal canal and a fourth portal for a fourth wall of the vaginal canal. In another aspect, a cylindrical speculum may be used having the spiral pattern 220 of portals and no rotation may be necessary. Additionally, numerous small pea sized amounts, (e.g., approximately 0.1 cc) of amniotic solution with or without hyaluronic acid may injected at external locations 110 in the lower external labia and lower external portion of the vaginal canal.

FIG. 2D is an anatomical diagram 200-D illustrating example internal locations in a spiral pattern 220 for injections, according to an aspect of the disclosure. Numerous small pea sized amounts, (approximately 0.1 cc) of amniotic solution with or without hyaluronic acid are injected in the spiral pattern 220 starting in the proximal portion of the vaginal canal and progressing distally along the anterior, posterior and lateral sidewalls of the vaginal canal. In this example, the injections are limited to the vaginal canal only and not the external labia.

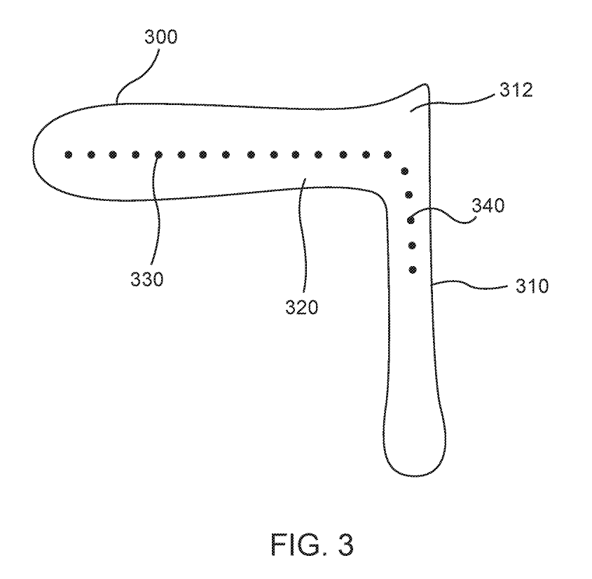

FIG. 3 illustrates an example of a vaginal speculum 300 according to an aspect of the disclosure. The vaginal speculum 300 may include a handle 310 and a longitudinal member 320. The handle 310 may be located near an opening 312 at a proximal end and extend transverse to the longitudinal member. The handle may provide a grip for a medical practitioner. In an aspect, the handle 310 may be used to expand the speculum 300.

The longitudinal member 320 may be adapted for insertion into the vaginal canal. The longitudinal member may have a curved wall having an external surface that contacts the vaginal canal and an interior surface. The interior surface may be accessible via the opening 312 of the speculum. The vaginal speculum 300 may take the form of other specula known in the art. For example, the vaginal speculum 300 may be a single bladed speculum, bivalve speculum, three bladed speculum, or cylindrical speculum. The longitudinal member 320 may be a blade or cylindrical portion of a speculum known in the art. The speculum 300 may be made from metal or plastic. In an aspect, a disposable speculum made of transparent plastic is preferable for visualization and sanitation.

The speculum 300 includes multiple needle portals 330 arranged in various walls of the speculum 300. In an aspect, a first plurality of needle portals 330 are arranged along the longitudinal member (e.g., blade or cylinder) of the speculum 300 in a straight line and a second plurality of needle portals 340 are arranged along the handle 310. Each needle portal 330, 340 may extend from an interior surface of a wall of the longitudinal member through to the external surface of the wall. Accordingly, each needle portal 330, 340 may be a through passage. The diameter of the needle portal 330, 340 may be sufficient to accommodate a range of needle sizes (e.g., 22-25 gauge). The opening of the needle portal 330, 340 on the inner surface of the wall may form a stop for the needle. A longitudinal stop may be attached to the needle such that a desired length of the needle extends past the stop. In an aspect, the body of the syringe may serve as the longitudinal stop. The diameter of the needle portal 330, 340 may not allow stop or the body of the syringe to pass. For example, the diameter of the needle portal may be 1-2 mm. When the needle is inserted through a needle portal 330, the longitudinal stop contacts the inner surface of the speculum and prevents further insertion of the needle. In an aspect, the desired depth of the injection is 4 mm to 6 mm. Accordingly, the needle portals 330, 340 may provide a guide such that the needle is inserted to the proper depth. The needle portals 330 may prevent accidental puncturing of nearby organs.

In an aspect, the needle portals 330 may be arranged according to any of the injection patterns described above with respect to FIGS. 2A-2D. For example, the needle portals 330 may be evenly spaced. The distance between the needle portals 330 may be 1 cm-1.5 cm, and each needle portal 330 may be used for an injection according to the selected pattern. In another example, the distance between the needle portals 330 may be approximately 0.5 cm and he medical professional may select which needle portals 330 to use, for example, every other needle portal 330 or every third needle portal 330. The needle portals 340 may be arranged according to any of the external injection patterns described above with respect to FIGS. 1A-1D. For example, the needle portals 340 may be evenly spaced at a distance of 0.5 cm-1.5 cm. The needle portals 340 may also extend circumferentially around all or part of the opening 312

In an aspect, each of the needle portals 330 located in the longitudinal member 320 may be angled toward the opening 312 of the speculum 300. Accordingly, needle portals 330 near the opening 312 may be approximately perpendicular to the interior surface of the speculum 300, whereas needle portals located at the distal end of the longitudinal member 320 may be angled at approximately 30 degrees. Accordingly, a needle inserted through the opening 312 may pass straight through each of the needle portals 330.

This written description uses examples to disclose the invention, including the preferred embodiments, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Aspects from the various embodiments described, as well as other known equivalents for each such aspect, can be mixed and matched by one of ordinary skill in the art to construct additional embodiments and techniques in accordance with principles of this application.

1. A method of vaginal rejuvenation comprising:
providing an injectable solution comprising amniotic membrane allograft; and
injecting the solution into a human female in a plurality of locations in a vaginal canal and introitus area to a depth immediately below a vaginal epithelium.

2. The method of claim 1, wherein the injectable solution further comprises hyaluronic acid.

3. The method of claim 1, wherein the injectable solution further comprises saline.

4. The method of claim 1, further comprising:
inserting a speculum into the vaginal canal, wherein the speculum comprises multiple spaced openings extending through at least one blade of the speculum,
wherein the injecting comprises inserting a needle through a respective one of the evenly spaced openings into each of the plurality of locations.

5. The method of claim 4, wherein inserting the needle comprises inserting the needle through the respective one of the spaced openings until a stop on the needle abuts a surface of the speculum.

6. The method of claim 5, wherein the needle extends to the depth immediately below the vaginal epithelium when the stop abuts the surface of the speculum.

7. The method of claim 1, wherein the injecting comprises injecting between approximately 0.1 cubic centimeters (cc) and 0.5 cc of the solution at each location.

8. The method of claim 7, wherein the injecting comprises injecting between approximately 0.1 cc and 0.2 cc.

9. The method of claim 1, wherein a distance between each location is approximately 1 cm to 4.0 cm.

10. The method of claim 1, wherein the distance between each location is approximately 1 cm to 1.5 cm.

11. The method of claim 1, further comprising injecting between 0.2 and 0.5 cc of the solution into a Gräfenberg spot of the human female.

12. The method of claim 1, further comprising preparing the vaginal canal with a cleansing scrub.

13. The method of claim 1, further comprising packing the vaginal canal with a lubricated vaginal packing and leaving the vaginal packing in place for at least 15 minutes.

14. A speculum comprising:
at least one longitudinal member extending from an opening along a longitudinal axis and having a wall that curves about the longitudinal axis having an inner surface and an outer surface, wherein the longitudinal member comprises multiple spaced portals extending through the wall.

15. The speculum of claim 14, wherein the multiple spaced portals are each spaced approximately 0.5 cm apart.

16. The speculum of claim 14, wherein the multiple spaced portals are arranged along a longitudinal line.

17. The speculum of claim 14, further comprising a handle portion including a second plurality of portals extending through the handle.

18. The speculum of claim 17, wherein the second plurality of portals allow injections through the second plurality of portals into an external labia.

19. A kit for performing a vaginal rejuvenation procedure, comprising:
a speculum having at least one longitudinal member extending from an opening along a longitudinal axis and having a wall that curves about the axis having an inner surface and an outer surface, wherein the longitudinal member comprises multiple spaced portals extending through the wall; and
an injectable solution comprising amniotic membrane allograft.

20. The kit of claim 19, further comprising a syringe.
